(19) United States
(12) Patent Application Publication
(10) Pub. No.: US 2025/0275948 A1
(43) Pub. Date: Sep. 4, 2025

(54) (N-[4-(2,3-DIHYDRO-1H-INDEN-5-YL)-1,3-THIAZOL-2-YL]-2-[4-METHYL-4-(NAPHTHALEN-2-YL)-2,5-DIOXOIMIDAZOLIDIN-1-YL]ACETAMIDE AS STABILIZER OF CLOCK:BMAL1 INTERACTION FOR THE TREATMENT OF CIRCADIAN RHYTHM DISEASES AND DISORDERS

(71) Applicant: KOC UNIVERSITESI, Istanbul (TR)

(72) Inventors: Halil KAVAKLI, Istanbul (TR); Safak ISIN, Istanbul (TR)

(73) Assignee: KOC UNIVERSITESI, Istanbul (TR)

(21) Appl. No.: 18/860,727

(22) PCT Filed: Apr. 29, 2022
(86) PCT No.: PCT/TR2022/050397

§ 371 Date: Oct. 28, 2024

(51) Int. Cl. A61K 31/427; G01N 33/50

(52) U.S. Cl. CPC A61K 31/427; G01N 33/5023

(57) ABSTRACT

An N-[4-(2,3-dihydro-1H-inden-5-yl)-1,3-thiazol-2-yl]-2-[4-methyl-4-(naphthalen-2-yl)-2,5-dioxoimidazolidin-1-yl]acetamide compound and related derivatives are provided. The compound is a CLOCK:BMAL1 complex-binding small molecule and also a stabilizer of CLOCK:BMAL1 interaction, and is therefore useful, as pharmaceutical agent, especially in the treatment and/or prevention of disorders associated with the circadian rhythm. The disorders include a sleep disorder, mood disorder or aging and diseases related with altered circadian amplitude.

### CROSS REFERENCE TO THE RELATED APPLICATIONS

This application is the national phase entry of International Application No. PCT/TR2022/050397, filed on Apr. 29, 2022, the entire contents of which are incorporated herein by reference.

### TECHNICAL FIELD

The present invention discloses and claims (N-[4-(2,3-dihydro-1H-inden-5-yl)-1,3-thiazol-2-yl]-2-[4-methyl-4-(naphthalen-2-yl)-2,5-dioxoimidazolidin-1-yl]acetamide (formula I) as circadian locomotor output cycles kaput: brain and muscle arnt-like 1 (CLOCK:BMAL1) complex-binding small molecule and stabilizer of CLOCK:BMAL1 interaction, and method of using said compound formula I for treating disorders including aging, mood disorders, sleep disorders and diseases related with altered circadian amplitude. Pharmaceutical compositions comprising formula I and methods for the preparation of formula (I) are also disclosed and claimed.

### BACKGROUND

The circadian clock generates a 24-hour rhythm through which physiology and behavior are adapted to daily changes in the environment. Many biological processes like hormone secretion, and sleep-wake cycles are controlled by the circadian clock. Therefore, an innate malfunctioning of the circadian clock and the related pathways can cause various pathologies. Sleep disorders, altered metabolism, obesity, diabetes, mood disorders, cancer, and cardiovascular diseases are all linked with abnormal circadian rhythm.

At the molecular level, the clockwork of the cell involves several proteins that participate in positive and negative transcriptional/translational feedback loops (TTFL). BMAL1 and CLOCK are transcription factors that bind E-box elements (CACGTG) in clock-controlled genes including Period (Per) and Cryptochrome (Cry) and thereby exert a positive effect on circadian transcription. The mammalian PERIOD (PER) and CRYPTOCHROME (CRY) proteins form heterodimers that interact with casein kinase Iε (CKIε) and then translocate into nucleus where CRY acts as a negative regulator of BMAL1/CLOCK-driven transcription. Upon phosphorylation CRYs are ubiquitinated by E3 ubiquitin ligases e.g. FBXL3 and FBXL21, and directed to proteasome for the degradation. FBXL3 and FBXL21 act antagonistically on CRY to regulate its stability differentially in the cytosol and nucleus. In addition, there is a second feedback loop consists of retinoic acid receptor-related orphan receptors (RORs) and REV-ERBs which control the transcription of the Bmal1 gene and, in turn, regulate molecular clock.

Since the circadian system regulates several aspects of our physiology, it is not surprising that disturbed circadian rhythm can lead into diseases in human. It is, therefore, essential to find small molecules to correct disturbed circadian rhythm. There are several studies have been carried out to find small molecules affect circadian rhythm based on phenotypic changes in the circadian rhythm of reporter cells using high-throughput screening assay. These studies result in identification several molecules affect different features of circadian rhythm. For example, a molecule (named as GSK4112) was shown to enhance REV-ERB's repressor function toward Bmal1 transcription and greatly altered circadian clock and metabolism. Another example, identification of KL001 molecule, increases stability of the CRYs and suppresses the gluconeogenesis. Alternatively, structure-based drug design approach can be utilized to design small molecules by using the available crystal structure core clock proteins. The crystal structure of the CRY-FBXL3 revealed the critical region on CRY for the FBXL3 interaction.

In the international patent document WO2018132383A1, small molecule agents that disrupt CRY1-CLOCK-BMAL1 ternary complexes are disclosed. According to this invention, these disrupting agents bind to the secondary pocket of CRY1 and in this way inhibit interaction between the secondary pocket and the CLOCK PAS-B domain. Agents of interest herein are small molecules, polymer, peptides, polypeptides.

Despite advances in drug discovery directed to identifying molecule interactions of CLOCK and/or BMAL1 protein activity, there is still a scarcity of compounds that are both potent, efficacious, and selective stabilizer of CLOCK:BMAL1 interaction. Furthermore, there is a scarcity of compounds effective in the treatment and/or prevention of disorders associated with the circadian rhythm. These needs and other needs are satisfied by the present invention.

The present invention relates to the therapeutic potential of CLOCK-BMAL1 for the treatment of Jet-lag and related diseases with clash of internal and external environmental clock. When someone travel between two or more time zones, they may experience jet lag. Jet lag happens when your internal clock (circadian rhythms), which regulates your sleep-wake cycle, is out of sync with the time in your new location because of traveling across numerous time zones. Clock in each cell works differently compared external time. Considering the CLOCK and BMAL1 are the core clock responsible for generating the circadian rhythm, a molecule regulates their activity, and, in turn, rest internal clock can be used for the treatment of jet-lag. Even though there are treatment methods such as exposure to bright light or melatonin intake, their effectiveness highly depends on the time they were used, and the time length of the new environment.

### SUMMARY

According to a first aspect of the invention there is provided a CLOCK:BMAL1 complex-binding compound of formula I or pharmaceutically acceptable salts thereof.

Accordingly, a broad embodiment of the invention is directed to a CLOCK:BMAL1 complex-binding compound of formula I:

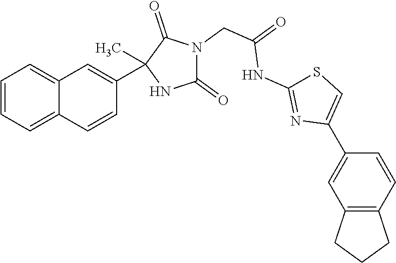 [SMILES: CC1(c2ccc3ccccc3c2)NC(=O)N(CC(=O)Nc2nc(-c3ccc4c(c3)CCC4)cs2)C1=O]

The other aspect of the present invention is to provide a CLOCK:BMAL1 complex-binding compound for treating and/or preventing a circadian rhythm associated diseases or disorders, wherein the compound is characterized by stabilizing CLOCK's interaction with BMAL1. The compound binds to CLOCK:BMAL1 complex, and it also stabilizes the interaction between these two binding partners which results in nuclear accumulation of CLOCK and BMAL1 proteins.

In a further aspect, the present invention relates to a CLOCK:BMAL1 complex-binding compound of the invention for use in increasing strength of positive loop mediated by increase in the CLOCK:BMAL1 level in nucleus and also decreased amplitude and increased period is the result of increasing the amount of CLOCK and BMAL1 in the nucleus. This is because the molecule changes the stoichiometric amount of the BMAL1 and CLOCK in the nucleus. Uses of the molecule as therapeutic molecule will enable rest circadian clock and, in turn, adjust body clock to the external environmental clock.

Another aspect of the present invention is to provide a CLOCK:BMAL1 complex-binding compound capable of controlling circadian rhythm and reducing the circadian rhythm amplitude.

Another embodiment of the present invention relates to a method for identifying a compound for stabilizing the interaction between CLOCK and BMAL1.

The invention can be used for the preparation of a medicament useful in the treatment and/or prevention of disorders due the stabilization of CLOCK:BMAL1 complex interaction. Yet another objective of the present invention is to provide a pharmaceutical composition comprising a pharmaceutical carrier and a therapeutically effective amount of a compound of formula I, or a pharmaceutically acceptable salt thereof.

Moreover, the present invention the therapeutic potential of CLOCK-BMAL1 for the treatment of Jet-lag and related diseases with clash of internal and external environmental clock. Even though there are treatment methods such as exposure to bright light or melatonin intake, their effectiveness highly depends on the time they were used, and the time length of the new environment. Because higher doses of Formula I (CLK95) completely suppress the endogenous clock, resynchronization of the internal clock to the time of the new environment can be efficiently operated.

This object and other objects of this invention become apparent from the detailed discussion of the invention that follows.

### BRIEF DESCRIPTION OF THE DRAWINGS

The present invention is illustrated in the accompanying figures wherein;

FIGS. 1A-1B are illustrations of Formula I (CLK95) docking result to CLOCK:BMAL1 complex. (FIG. 1A) Best binding model of CLK95 to CLOCK:BMAL1 complex with surface representation of CLOCK:BMAL1 binding sites of CLK95. CLK95 binds to CLOCK:BMAL1 complex with-9.19 kcal/mol energy. (FIG. 1B) Chemical Structure of CLK95 with predicted interacting residues of CLOCK:BMAL1 complex

FIGS. 2A-2B are illustrations of non-toxic doses of Formula I (CLK95) were determined using MTT assay. (FIG. 2A) Assessment of cell viability in U2OS Bmal1-dLuc cells. (FIG. 2B) Assessment of cell viability in NIH 3T3 Bmal1-dLuc cells. Cells were treated with indicated doses of CLK95 (0.25% DMSO), 48 hours after cell seeding. Cells were incubated for 48 hours with CLK95 doses, and cell viability was determined using Synergy H1 Microplate Reader (Bio-Tek Instruments) as previously discussed. All measurements were normalized against control (0.25% DMSO). Doses that showed ≥80% cell viability were selected as non-toxic doses. (Data represent the mean±SEM; n=3).

FIGS. 3A-3B are illustrations of Formula I (CLK95) that does not affect the half-life of LUCIFERASE. (FIG. 3A) Effect of CLK95 on LUCIFERASE half-life was assessed in HEK 293T cells that were transfected with pcDNA-Luc. Luciferase signal recorded after CLK95 (0.25% DMSO) treatment until it reached to a plateau (FIG. 3B) Graphical representation of LUC half-life quantification. (Data represent the mean±SEM; n=3).

FIGS. 4A-4B are illustrations of CLK95 specifically decreases the amplitude and increases the period. (FIG. 4A) Continuous monitoring of circadian oscillations in NIH3T3 Bmal1-dLuc cells using Lumicycle, Actimetrics. (FIG. 4B) Continuous monitoring of circadian oscillations in U2OS Bmal1-dLuc cells using Synergy H1. After seeding, cells were synchronized and treated with indicated doses of CLK95 (0.25% DMSO). Then, cells were placed in Lumicycle or Synergy H1 to record luminescence signals for 5-7 days with 10 minutes or 24 minutes intervals, respectively. The data of the first day were omitted. Data represent the mean±SEM; n=3. *p-value <0.05; **p-value <0.01; ***p-value<0.001.

FIGS. 5A-5D are illustrations of CLK95 effecting the circadian phenotype through CLOCK and BMAL1. (FIG. 5A) CLOCK and BMAL1 levels of wild type (WT), Clock KO, and Bmal1 KO MEF cells. Continuous monitoring of circadian rhythm after CLK95 treatment in (FIG. 5B) WT, (FIG. 5C) Clock knockout, and (FIG. 5D) Bmal1 knockout MEF cells. After cells were transduced with Bmal1-dLuc lentiviral particles to stably express Luc, they were treated with CLK95 and put in Lumicycle to record Luminescence signaling for 7 days with 10 minutes intervals. Data of first day is omitted. Data represent the mean±SEM; n=3. *p-value <0.05; **p-value <0.01; ***p-value<0.001.

FIGS. 6A-6E are illustrations of CLK95 specifically binding to CLOCK:BMAL1 complex. (FIG. 6A) Chemical structure of biotinylated-CLK95 (bCLK95). Pull-down assays conducted by employing overexpression systems that used (FIG. 6B) only CLOCK overexpressing, (FIG. 6C) only BMAL1 overexpressing, and (FIG. 6D) CLOCK:BMAL1 co-expressing HEK 293T lysates. (FIG. 6E) Whole cell lysate of U2OS cells were used to explore binding at endogenous level. While 20 μM bCLK95 was used as bait, 200 μM non-biotinylated CLK95 used as competitor. Data are representative for three independent studies for FIGS. 4A-4B and FIGS. 5B-5D.

FIGS. 7A-7B are illustrations of CLK95 increasing the nuclear localization of CLOCK and BMAL1. (FIG. 7A) Subcellular fractionation was performed on unsynchronized U2OS Bmal1-dLuc cells that were treated with 5 μM CLK95 (0.25% DMSO) and. CLOCK, BMAL1, CRY1, and PER2 proteins were immunoblotted. Protein levels were normalized to HISTONE H3 and α-TUBULIN levels. (FIG. 7B) Quantification of core-clock protein levels in nucleus and cytoplasm after CLK95 treatment. Data are mean±SEM; n=4, independent experiments. *p-value <0.05; **p-value <0.01; ***p-value<0.001.

FIGS. 8A-8E are illustrations of CLK95 increasing the interaction between CLOCK and BMAL1, and Arg126 of BMAL1 is essential for CLK95's binding to CLOCK:BMAL1 complex. (FIG. 8A) Interaction levels of CLOCK:BMAL1 were assessed under CLK95 presence via Co-IP assay. FLAG-BMAL1 predicated by anti-FLAG resin and the amounts of CLOCK it came with were determined with western blotting. (FIG. 8B) Graphical representation of the quantification of CLOCK:BMAL1 interaction after CLK95 treatment. Level of CLOCK proteins that were precipitated along with FLAG-BMAL1 were normalized to BMAL1 levels. (FIG. 8C) Co-IP assay were used to evaluate the interaction levels of CLOCK-R126A:BMAL1 under CLK95 treatment. (FIG. 8D) Graphical representation of the quantification of CLOCK-R126A:BMAL1 interaction after CLK95 treatment. (FIG. 8E) Functional evaluations of R126 BMAL1 in comparison to WT BMAL1. Evaluations were performed by using transactivation assay on HEK 293T cells. Data are mean±SEM; n=3. *p-value <0.05; **p-value <0.01; ***p-value<0.001.

FIGS. 9A-9B are illustrations of CLOCK and BMAL1 protein levels increased after CLK95 treatment. (FIG. 9A) Unsynchronized U2OS Bmal1:dLuc cells were treated with indicated doses of CLK95. 24 hours after molecule treatment, cells were harvested, and western blot was performed. Protein levels were normalized to β-ACTIN levels. (FIG. 9B) Quantification of protein levels in unsynchronized U2OS Bmal1:dLuc cells. Data represents mean±SEM; n=3, independent experiments. *p-value <0.05; **p-value <0.01.

FIGS. 10A-10B are illustrations of alterations at protein levels after CLK95 treatment in synchronized U2OS Bmal1:dLuc cells. (FIG. 10A) U2OS Bmal1:dLuc cells that were synchronized with 0.1 μM dexamethasone, were treated with 5 μM CLK95 (0.25% DMSO). 24 hours after molecule treatment, cells were harvested at indicated times and western blot was performed. Protein levels were normalized to β-ACTIN levels. (FIG. 10B) Quantification of protein levels of synchronized U2OS Bmal1:dLuc cells. Data represents mean±SEM; n=3, independent experiments. *p-value <0.05; **p-value <0.01.

FIG. 11 is an illustration of the effect of CLK95 on transcription levels of core-clock and clock-control genes in unsynchronized U2OS Bmal1:dLuc cells. Quantification of mRNA levels was done using 2-ΔΔCt method. Normalizations were done using RPLP0 levels. Data represent mean±SEM; n=3, independent experiments. *p-value <0.05.

FIGS. 12A-12B are illustrations of the effect of CLK95 on core-clock proteins in mice liver. (FIG. 12A) Mice that treated with CLK95 (50 mg/kg) or vehicle sacrificed, and total protein levels of core-clock proteins were immunoblotted. (FIG. 12B) Representation of quantification of core-clock proteins in mice liver. Protein levels were normalized against β-ACTIN levels. Data represent mean±SEM; n=4 for control mice and n=5 for mice treated with CLK95. *p-value <0.05.

FIGS. 13A-13B are illustrations of effect of CLK95 on subcellular localization of core-clock proteins in mice liver. (FIG. 13A) Mice that subjected to CLK95 (50 mg/kg) or vehicle sacrificed and, livers of animals were harvested. After subcellular fractionation was performed, levels of core-clock proteins were immunoblotted in nuclear and cytosolic fractions. (FIG. 13B) Quantification of core-clock proteins in nucleus and cytoplasm. Protein levels were normalized against HISTONE H3 or α-TUBULIN levels. Data represent mean±SEM; n=3 for control mice and n=3 for mice treated with CLK95. *p-value <0.05; **p-value <0.01.

FIG. 14 is an illustration of effect of CLK95 on transcription levels of core-clock and clock-controlled genes in mice liver. 4 hours after molecule administration (50 mg/kg), mice were sacrificed, and livers of animals were harvested. mRNA levels were assessed using 2-ΔΔCt method. Normalizations were done using Gapdh levels. Data represent mean±SEM; n=3 for control mice and n=3 for mice treated with CLK95. *p-value <0.05.

The pieces/parts on the figures are numbered and the information corresponding the numbers is presented below:
IRoMAL1. Interacting Residues of BMAL1
IRoCLOCK. Interacting Residues of CLOCK

### DETAILED DESCRIPTION OF THE EMBODIMENTS

In the present invention, it is aimed to discover a novel CLOCK:BMAL1 complex binding small compound (Formula I; the compound) using a structure-based approach. As a result, the interaction of a CLOCK:BMAL1 complex-binding compound with the complex has been predicted in silico and then demonstrated experimentally.

It is found that the compound alters the circadian rhythm. Detailed in silico analyses suggest that the compound specifically binds to CLOCK:BMAL1 complex. In vitro and in vivo studies were performed to fully characterize the effect of the compound.

The CLOCK:BMAL1-binding compound was identified that increases the interaction between CLOCK and BMAL1, stabilizes the CLOCK:BMAL1 complex, increases the CLOCK and BMAL1 protein levels. The present invention relates to a CLOCK:BMAL1 complex-binding compound of the invention for use in increasing strength of positive loop mediated by increase in the CLOCK:BMAL1 level in nucleus and also decreased amplitude and increased period is the result of increasing the amount of CLOCK and BMAL1 in the nucleus.

Furthermore, it was discovered that, as a result of this binding interaction, the nuclear location of CLOCK and BMAL1 is regulated by the compound (Formula I) resulting in increase of the protein level in nucleus. An increase in nuclear CLOCK and BMAL1 leads the stabilization of the positive arm of the TTFL and, in turn, decreased amplitude and increased period is the result of increasing the amount of CLOCK and BMAL1 in the nucleus.

The present invention relates to a CLOCK:BMAL1 complex-binding compound (formula I).

Unless specified otherwise, the term “formula I” or “compound” or “CLK95” refers to compounds of formula I, prodrugs thereof, salts of the compound and/or prodrug, hydrates or solvates of the compound, stereoisomers, tautomers, isotopically labeled compounds, polymorphs, and derivatives of pharmacophore formula I.

It is an object of this invention to provide a CLOCK:BMAL1 complex-binding compound (named CLK95) having the chemical name (N-[4-(2,3-dihydro-1H-inden-5-yl)-1,3-thiazol-2-yl]-2-[4-methyl-4-(naphthalen-2-yl)-2,5-dioxoimidazolidin-1-yl]acetamide (IUPAC) as stabilizer of CLOCK:BMAL1 complex and so enhancer of transcriptional activity of CLOCK:BMAL1.

In accordance with the purpose(s) of the invention, as embodied and broadly described herein, the present invention relates to a CLOCK:BMAL1-binding compound, capable of stabilizing the interaction between CLOCK and BMAL1 by binding to CLOCK:BMAL1 complex. In this regard, the invention relates to a compound having the following formula I:

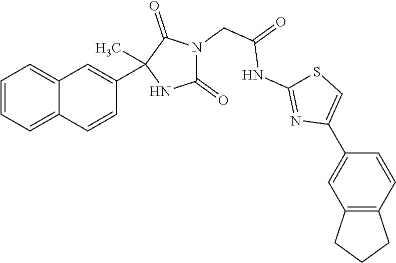 [SMILES: CC1(c2ccc3ccccc3c2)NC(=O)N(CC(=O)Nc2nc(-c3ccc4c(c3)CCC4)cs2)C1=O]

or a pharmaceutically acceptable salt thereof.

In a further aspect, the present invention relates to a compound (formula I) that binds to a CLOCK:BMAL1 complex and positively modulate CLOCK:BMAL1 activity.

The present invention relates to a novel CLOCK:BMAL1-binding small molecule that affects the nuclear translocation of the CLOCK and BMAL1 proteins into nucleus through the modulation of the BMAL1 and CLOCK interaction and thus alter the circadian rhythm. CLOCK and BMAL1 are dynamically interacting. When formula I is present, it binds to CLOCK:BMAL1 complex and increases CLOCK and BMAL1 interaction. Upon binding of formula I to CLOCK:BMAL1 complex, the nuclear translocation of the CLOCK and BMAL1 into nucleus increased. Thus, formula I decreases the amplitude of the circadian rhythm.

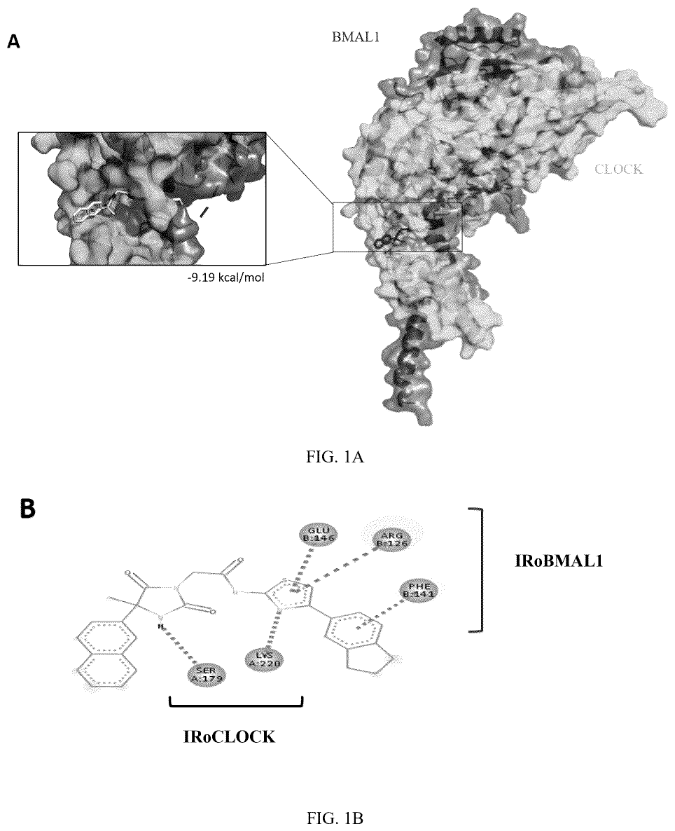

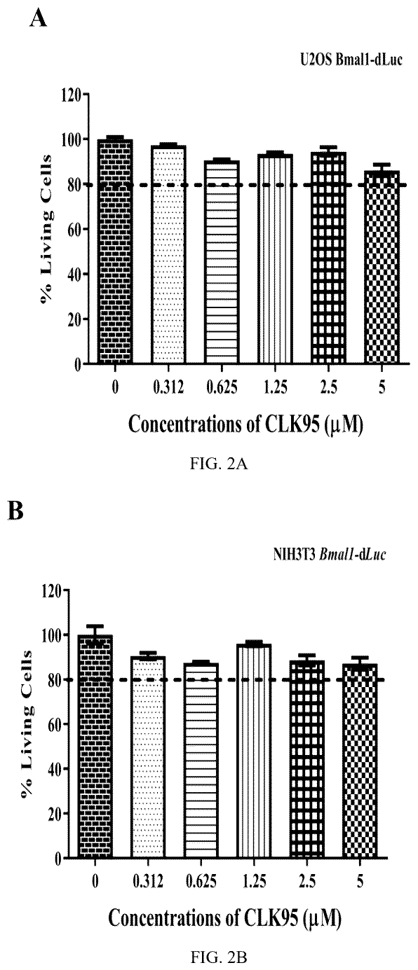

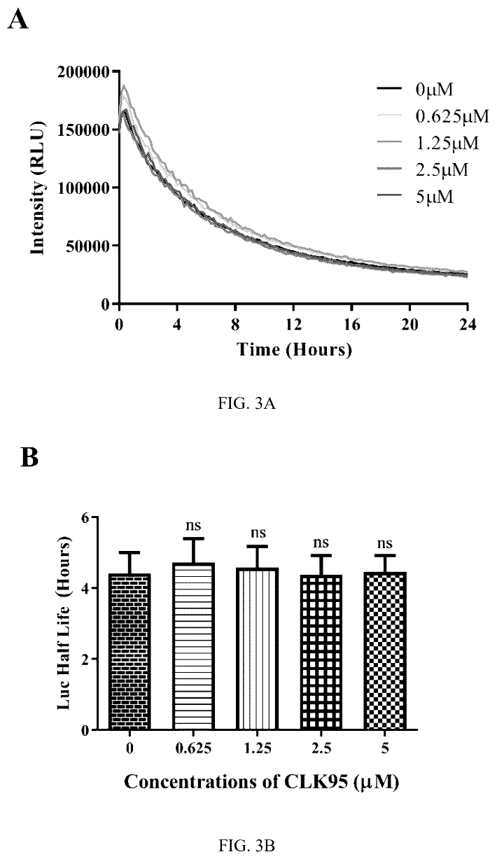

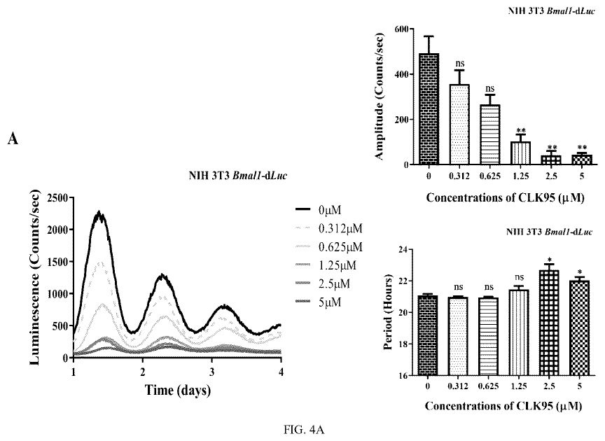

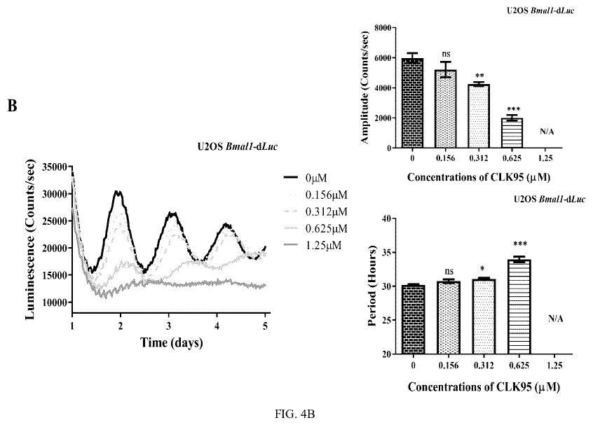

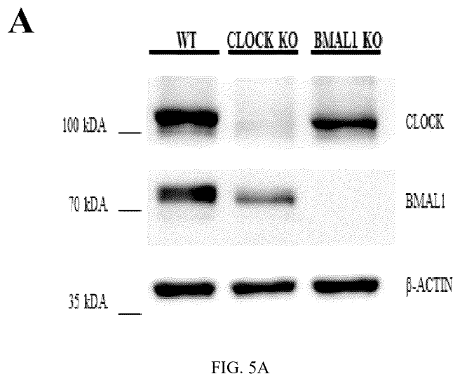

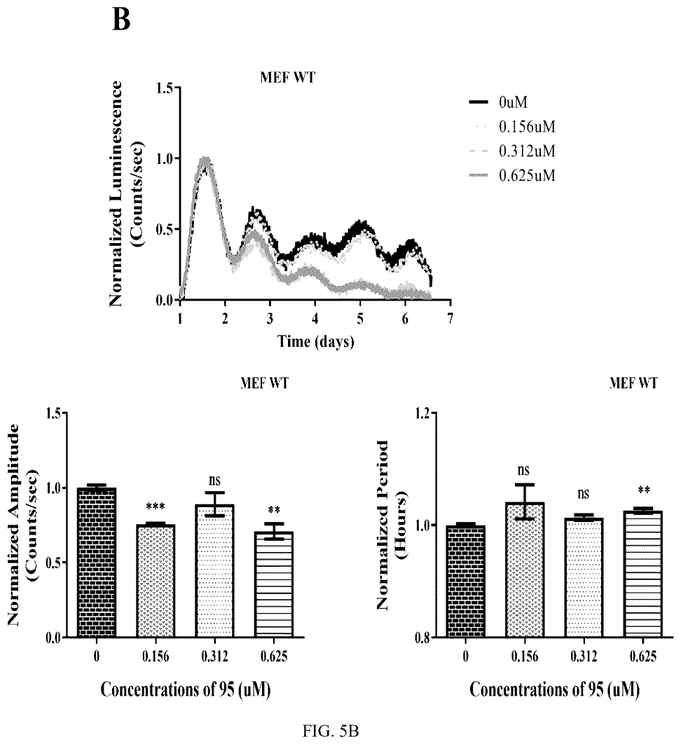

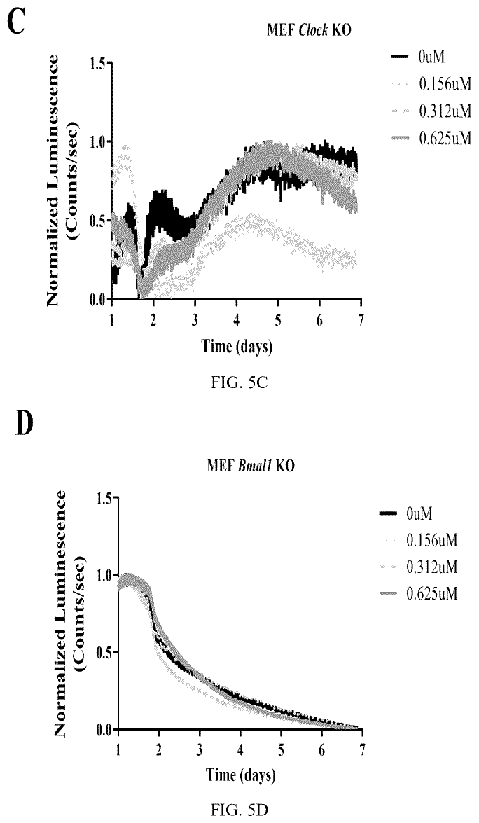

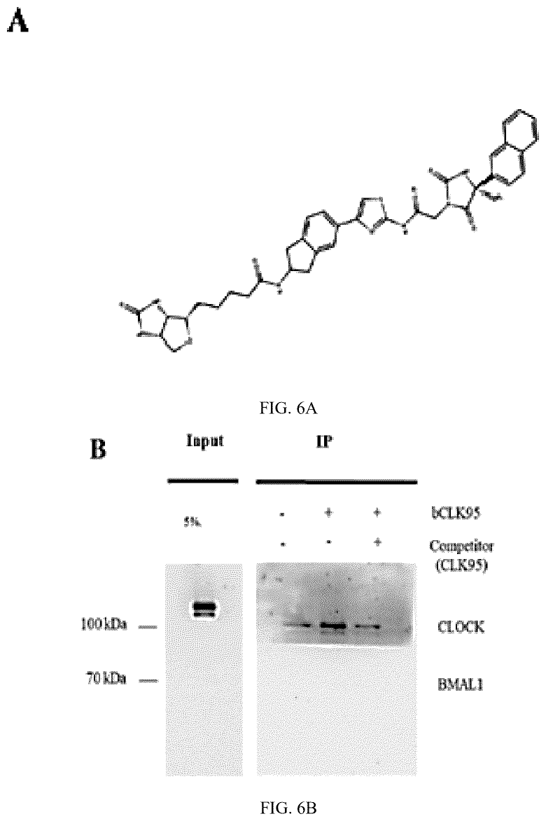

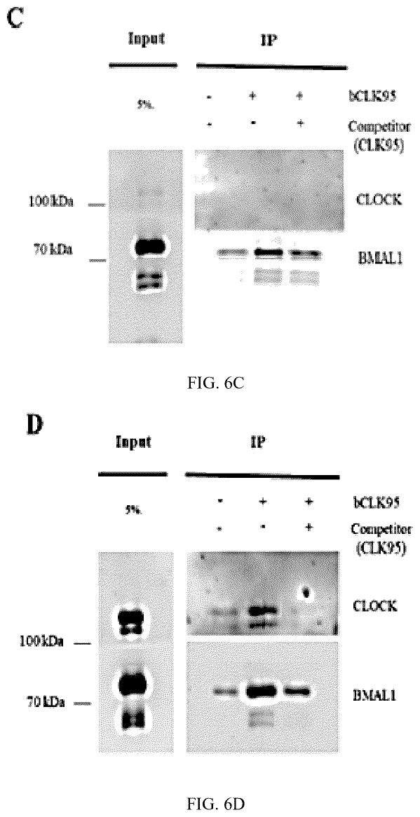

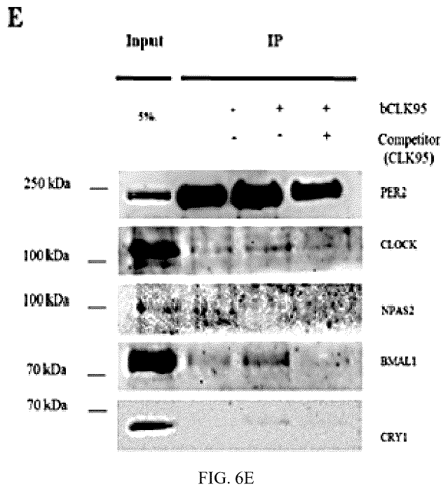

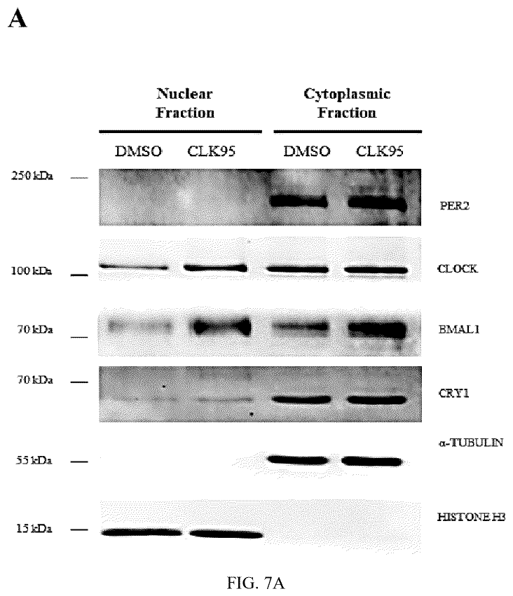

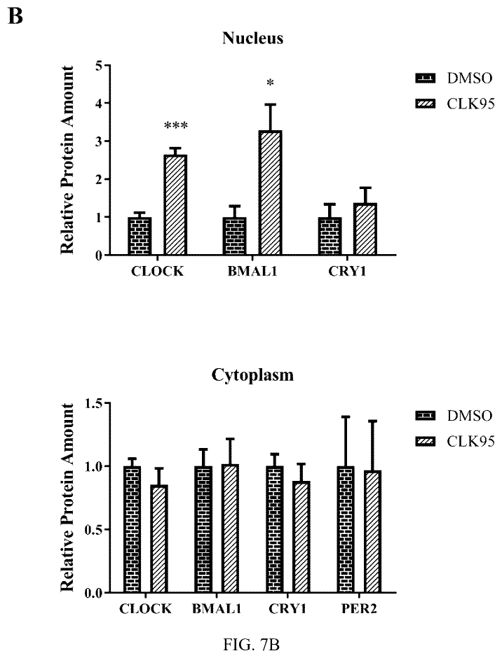

In one embodiment of the invention, the disclosed compound exhibits selectivity and high affinity for the CLOCK:BMAL1 complex. Thus, the complex stabilization activity of the compound is potent. Formula I binds to CLOCK:BMAL1 complex with-9.19 kcal/mol energy. Chemical Structure of the compound with predicted interacting residues of CLOCK:BMAL1 complex is shown in FIGS. 1A-1B.

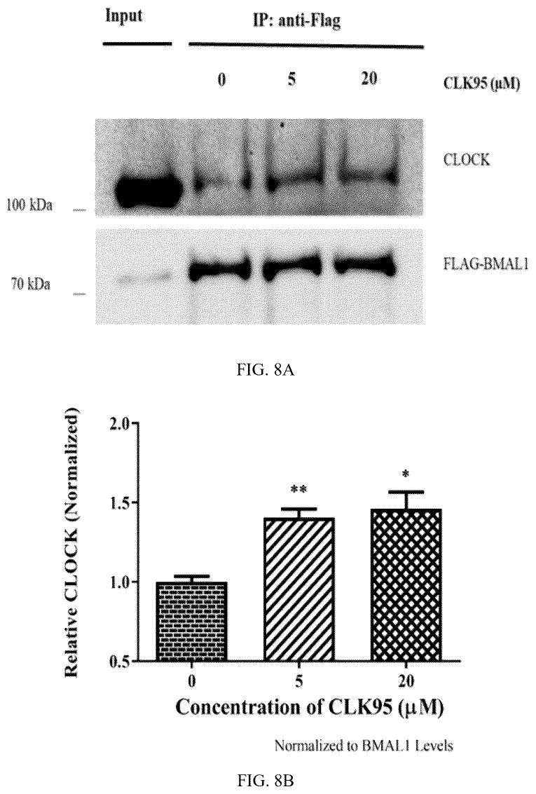

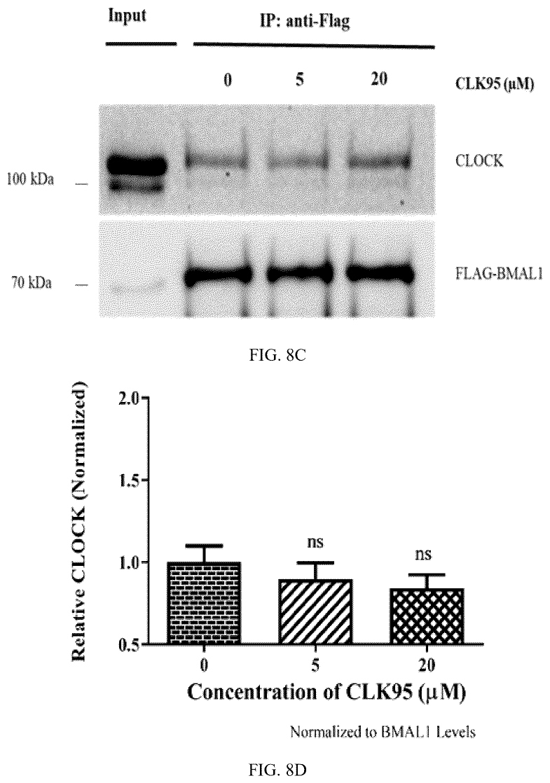

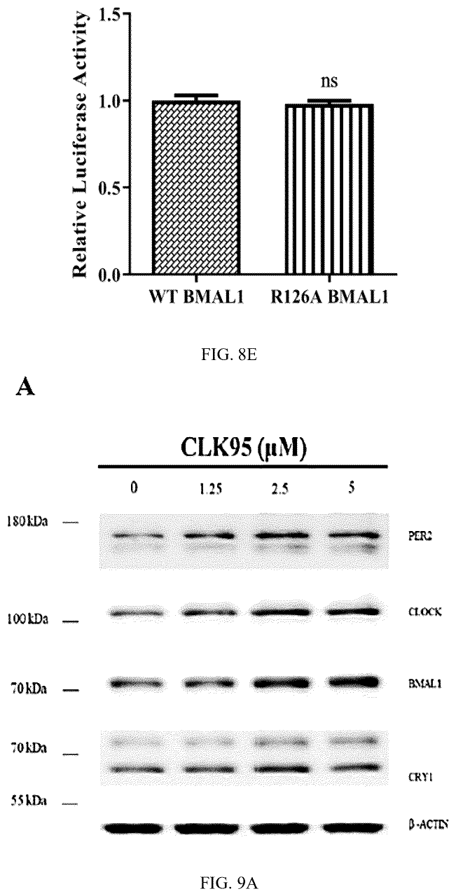

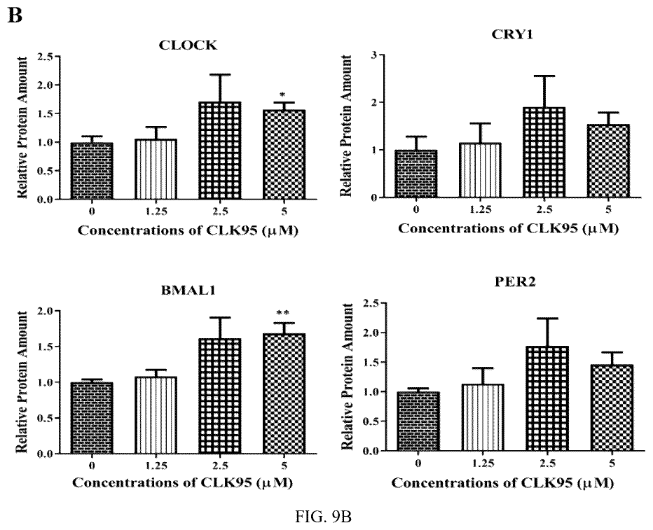

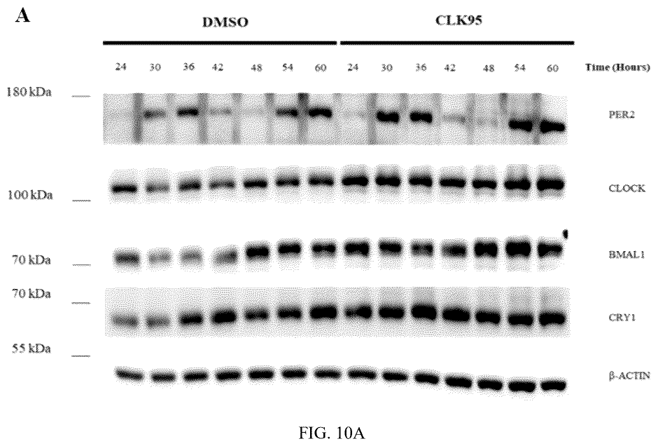

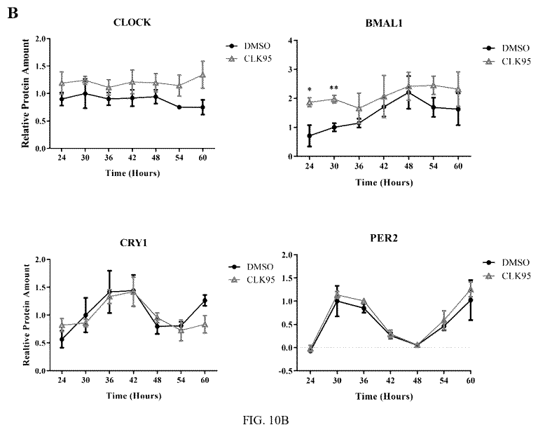

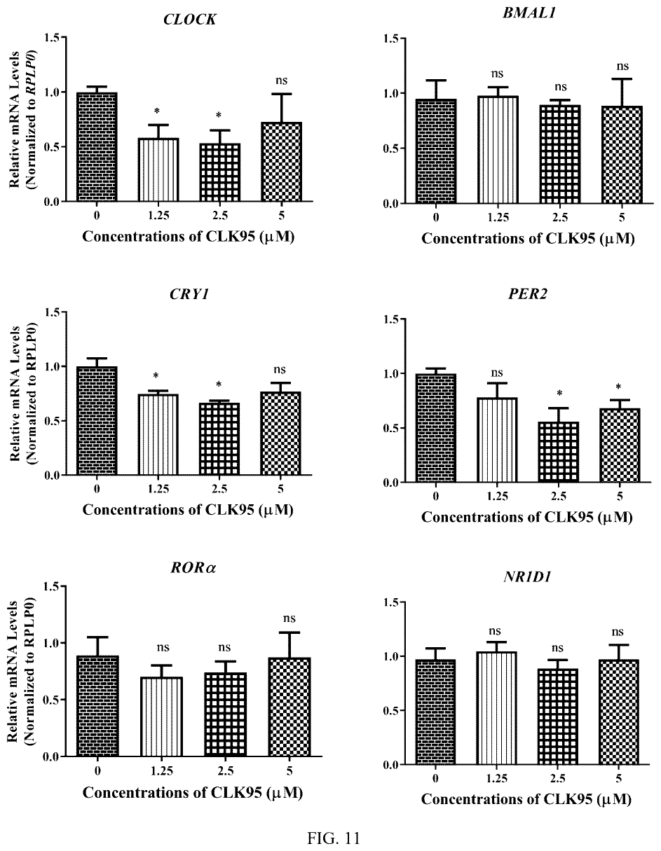

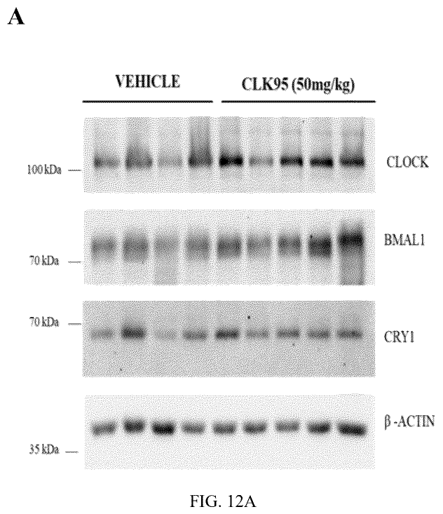

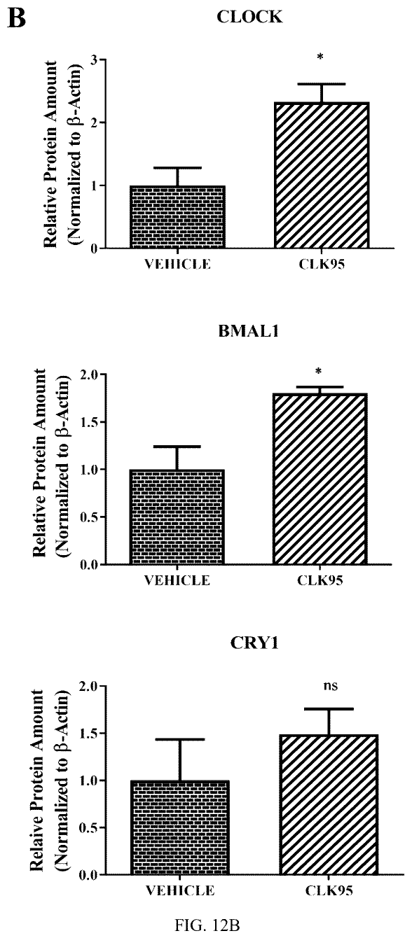

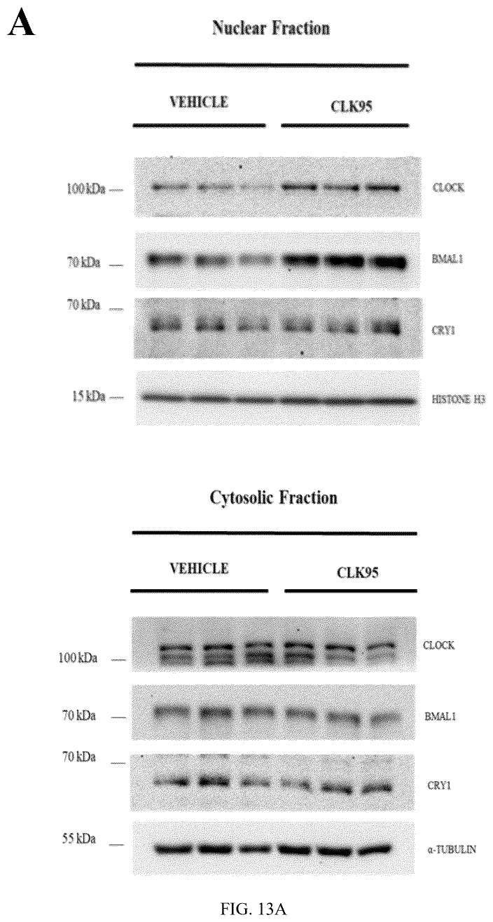

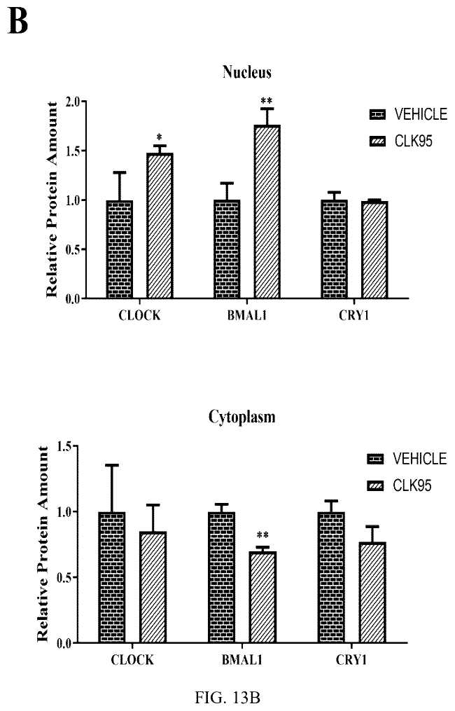

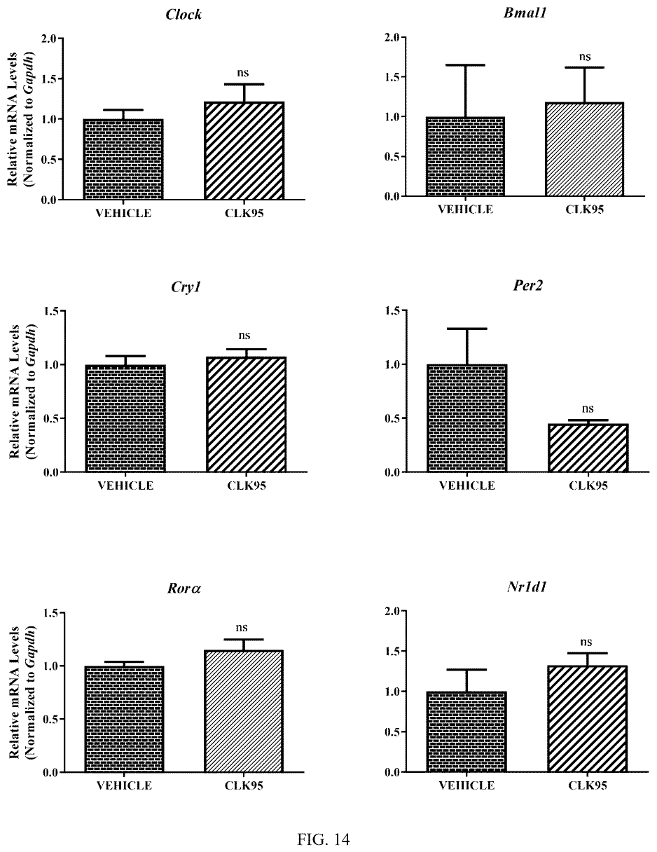

In certain aspects, the compound interacts with certain amino acid residues of CLOCK and BMAL1 proteins. For CLOCK protein these interacting residues are Ser 179 and Lys 220 (IRoCLOCK). For BMAL1 protein these interacting residues are Glu 146, Arg 126, and Phe 141 (IRoBMAL1) (FIGS. 1A-1B). CLK95 increases the interaction between CLOCK and BMAL1, and Arg126 of BMAL1 is essential for CLK95's binding to CLOCK:BMAL1 complex (FIGS. 8A-8E).

Binding of the compound to CLOCK:BMAL1 increases the interaction between the CLOCK and BMAL1, thereby stabilizing an existing CLOCK:BMAL1 heterodimer complex. This stabilization results in an increase in CLOCK:BMAL1 dimerization and, in turn, the translocation of CLOCK and BMAL1 into the nucleus through the modulation of the BMAL1 and CLOCK interaction.

As used herein, the term “stabilize”, “stabilization” or “stabilizing” refers to a significant increase in the baseline activity of a biological activity or process such as CLOCK:BMAL1 interaction.

The term “a therapeutically effective amount” of a compound of the present invention refers to a non-toxic and sufficient amount of the compound of the present invention that will elicit the biological or medical response of a subject, for example, increase or stabilization of the protein:protein interaction.

All of the various embodiments of the present invention as disclosed herein relates to methods of treating and/or preventing various diseases and disorders as described herein. As stated herein the compound used in the method of this invention are capable of increasing and/or stabilizing the effects of CLOCK:BMAL1 heterodimer complex.

The invention further provides methods for the treatment or prevention of circadian rhythm related disorders and diseases. Non-limiting examples of circadian rhythm disorders include aging, sleep disorders, altered metabolism (metabolic syndromes), obesity, diabetes, mood disorders, and cardiovascular diseases. Mood disorders including major depressive disorder, bipolar I disorder; sleep disorders including circadian rhythm sleep disorders such as shift work sleep disorder, jet lag syndrome, advanced sleep phase syndrome, non-24-hour sleep-wake syndrome, irregular sleep-wake rhythm, and delayed sleep phase syndrome.

Moreover, the invention relates to a pharmaceutical composition comprising such compounds, uses and methods of use for such compounds in the treatment and/or prevention of disorders associated with the circadian rhythm. In other embodiment of the present invention, a pharmaceutical composition comprising the compound is useful in the treatment and/or prevention of circadian rhythm related diseases and disorders due the increase of CLOCK:BMAL1 interaction and stabilization of the complex.

The present invention relates to pharmaceutical compositions comprising a pharmaceutical carrier and a therapeutically effective amount of a compound of formula I, or a pharmaceutically acceptable salt thereof.

The present invention also relates to pharmaceutical compositions to treat and/or prevent a CLOCK:BMAL1-mediated disorder, such as different metabolic syndromes during in aging, mood disorders and diseases related to dampened the circadian rhythm.

In one aspect, the disclosure relates to a method for the manufacture of a medicament for increasing CLOCK and BMAL1 interaction and stabilization of the complex in a mammal comprising combining a therapeutically effective amount of a disclosed compound with a pharmaceutically acceptable carrier or diluent.

The present invention provides a method for identifying a compound for increasing CLOCK and BMAL1 interaction and stabilization of the complex. The method can be established using systems for pharmaceutical screening that are well known in the art.

In one aspect, the present invention provides a method for identifying a compound that increases the interaction between CLOCK and BMAL1, wherein the method comprises contacting a compound with CLOCK:BMAL1 complex under conditions allowing for the interaction of the compound with CLOCK:BMAL1, and determining whether the compound increases the interaction between CLOCK and BMAL1 by using a system that uses a signal and/or a marker generated by the interaction between CLOCK and BMAL1 to detect presence or absence or change of the signal and/or the marker. The term “signal” as used herein refers to a substance that can be detected directly by itself based on the physical properties or chemical properties thereof. The term “marker” refers to a substance that can be detected indirectly when the physical properties or biological properties thereof are used as an indicator.

These examples are intended to representative of specific embodiments of the invention and are not intended as limiting the scope of the invention.

### SPECIFIC EMBODIMENTS

In these embodiments, a structure-based design was applied to find small molecules that specifically bind to the circadian core CLOCK and/or BMAL1 proteins. After identifying candidate molecules by virtual screening, experimental studies lead to discover a compound (formula I) that specifically binds to CLOCK:BMAL1 complex. It regulates the interaction between CLOCK and BMAL1 by interfering with the translocation of CLOCK and BMAL1 into the nucleus both in vivo and in vitro. Further studies indicated that formula I decreases the amplitude and increases the period of the circadian rhythm at the cellular level.

In detail, the present invention relates to discovery a molecule (CLK95) that stabilizes CLOCK-BMAL1 interaction using structure-based drug design and experimental approaches. CLK95 have shown promising results by increasing the stability of CLOCK-BMAL1 and destroying the circadian rhythm in Bmal1-dLuc U2OS cells.

### EXAMPLES

### Example 1 in Silico Search for Compounds that Interact with CLOCK:BMAL1 Complex

The initial structure of the CLOCK/BMAL1 complex protein is obtained from the Protein Data Bank (PDB ID 4F3L). Molecular dynamic (MD) simulations were carried out by NAMD software using CHARMM force field. First, the protein structure files for MD simulations were prepared by visual MD after removing crystallographic water molecules and adding hydrogen atoms using AutoDockTools4. Using the psfgen package, atom and residue names were replaced with the ones recognized by NAMD. Then, the structure was dissolved in a water-box and the system was ionized. In the first 10,000 steps of minimization, the backbone was fixed. Further 10,000 steps of minimization were performed on all atoms with no pressure control. Subsequently, the system was brought to physiological temperature (310 K) by 10 K increments with 10-ps simulation for each increment in which α-carbons were restrained. The constraint scaling decreases from 1 to 0.25 kcal/(mol Å²) in 0.25 increments, with each increment being 5,000 steps. Further 90,000 steps with zero constraint scaling were performed as the final part of energy minimization before the RMSD of protein was converged and stabilized. MD simulation for equilibrium was performed using the Langevin dynamics at 310 K with a damping coefficient of 5 ps⁻¹, 1 atm constant pressure, and the Langevin piston period and decay of 100 and 50 fs, respectively. The bonded interactions, the van der Waals interaction with 12 Å cutoff, and the long-range electrostatic interactions with the particle-mesh Ewald were considered in the calculations of the forces acting on the system. At the end, the RMSD of the CLOCK backbone with respect to its initial structure showed that the equilibrium was reached after 3-4 ns. The final structure of the CLOCK after 10 ns of simulation was used as the receptor for docking step.

After MD simulations, water molecules of the system were deleted and the PDBQT file of the receptor, CLOCK, was prepared using AutoDockTools4. The library of commercially available small molecules (by Ambinter) was filtered according to “Lipinski's Rule of Five” considering a maximum limit of one violation. Finally, ˜2 million small molecules were selected for docking. PDBQT files of ligands were prepared by means of an automated script in Python language. We used AutoDock Vina for estimating protein-ligand affinity and predicting the best binding conformations of the compounds. The search space was defined to include the whole CLOCK protein to perform blind docking. The exhaustiveness was set as the default. Ultimately, the compounds were ranked based on their binding affinities (kcal/mol). The protein-compound interactions of the top 500 hit compounds were visually examined by the Discovery Studio Visualizer. The threshold value for hydrogen bonding was set as 3.4 Å and the accessible surface area was created considering a radius of 1.4 Å for solvent molecules. In the process of selecting hit molecules, compounds with docking positions far from CLOCK:BMAL1 protein interfaces were eliminated. Diversity in both binding region and chemical structure and shape complementarity as other important factors contributing in protein-ligand interactions were considered.

### Example 2 Cell Culture

Human embryonic kidney cells (HEK 293T), human osteosarcoma cell line stably express destabilized luciferase under the control of the Bmal1 promotor (U2OS Bmal1-dLuc), NIH 3T3 cell line stably express destabilized luciferase under the control of the Bmal1 promotor (NIH 3T3 Bmal1-dLuc), wild-type mouse embryonic fibroblast cells (WT MEF), Clock-knockout mouse embryonic fibroblast cells (MEF Clock KO) and/or Bmal1-knockout mouse embryonic fibroblast cells (MEF Bmal1 KO) were used in different experimental setups. Cells were maintained at 37° C. under 5% CO2 in a 95% humidified incubator using D10 medium (Dulbecco's Modified Eagle Medium with high glucose supplemented with 10% heat-inactivated Fetal Bovine Serum (Thermo Scientific) and 100 μg/mL Penicillin/Streptomycin cocktail).

Subculturing was regularly performed when cells reached confluency, or the medium was depleted of nutrients. First, the old medium was aspirated, and the cells were washed with 5 mL 1×PBS. Then the cells were trypsinized with 1 mL Trypsin-EDTA (Multicell, Cat. No 325-043-EL) solution. After incubation with Trypsin-EDTA for 2 min to detach the cells, D10 medium was added to cells for trypsin inactivation. Subsequently, cells were split into new 10 cm cell culture dishes with a 1:6 ratio.

### Example 3 Cell Cytotoxicity Assay

MTT (3-(4,5-dimethylthiazol-2-yl)-2,5-diphenyltetrazolium bromide) assay was used to measure cell viability in NIH 3T3 Bmal1-dLuc and U2OS Bmal1-dLuc cells. To perform this calorimetric assay, cells were seeded as triplicates into a clear 96-well plate (4×103 cells/well) and incubated for 48 hours at 37° C. in a humidified incubator under 5% CO2. After incubation cells were treated with appropriate amounts of CLK95 (control cells were treated with 0.25% DMSO) and incubated for another 48 hours at 37° C. under 5% CO2. Then, cell media was replaced with MTT reagent (final concentration: 1 mg/mL)/DMEM mix. After 4 hours incubation, the media was exchanged with DMSO:EtOH (1:1) mixture to dissolve formazan salts to measure absorbance (A) of the wells at 600 and 690 nm by Synergy H1 Microplate Reader (Bio-Tek Instruments). The cell viability was calculated according to Equation 3.1. by using recorded absorbance values.

$$\begin{matrix}Equation 3.1 . Cell viability calculation formula & \\ \% Cell viability = \frac{( A 600 nm - A 690 nm ) sample}{( A 600 nm - A 690 nm ) DMSO} \times 100 . & \end{matrix}$$

### Example 4 Protein Degradation Assay

Protein degradation assay was executed in transiently transfected HEK 293T cells which were seeded as triplicates into an opaque 96-well plate (4×104 cells/well). To carry out this experiment, reverse transfection technique was employed. Depending on the experiment, cells were transfected with PEI (final concentration: 6 ng/μL) using either 4 ng pcDNA4-Luc plasmid or 50 ng pcDNA4-Bmal1:Luc with or without 250 ng pCMV-Sport6-CLOCK plasmids. For each condition, maximum plasmid amount was limited to 300 ng. If the total plasmid amount was below 300 ng, empty pCMV-Sport6 plasmid was used to achieve the desired plasmid amount. After reverse transfection, cells were treated with appropriate doses of CLK95 and incubated at 37° C. under 5% CO2. Next day, medium of the cells was supplemented with HEPES-NaOH (final concentration: 10 mM, pH 7.2) and Luciferin (final concentration: 0.4 mM) and further incubated for an hour at 37° C. under 5% CO2. Then, cells were treated with CHX (final concentration: 20 μg/ml) to inhibit protein synthesis. Finally, the plate is sealed with an optically clear film and placed to Synergy H1 Microplate reader to record luminescence readings every 15 minutes at 32° C. for 24 hours. To calculate the protein half-life, one-phase exponential decay function was used.

### Example 5 Real-time Monitoring of Circadian Oscillation

NIH 3T3 Bmal1-dLuc and U2OS Bmal1-dLuc cells were used for real-time monitoring of circadian oscillation. While LumiCycle 32 (Actimetrics, USA) was used for recording the luminescence signals of NIH 3T3 Bmal1-dLuc cells, Synergy H1 (BioTek, USA) was used for recording the luminescence signals of U2OS Bmal1-dLuc cells. NIH 3T3 Bmal1-dLuc cells were plated in 35 mm cell culture dish at density of 45×104 cell/plate and incubated overnight at 37° C. under 5% CO2. The next day, cell medium was replaced with 0.1 μM Dexamethasone (Sigma D-8893) containing DMEM to synchronize the cells. After 2 hours incubation, cell media was exchanged with appropriate amount of CLK95 containing recording medium (10 g low glucose DMEM powder (Sigma D2902), 3.5 mg/mL D(+) glucose powder (Sigma G7021), 0.35 mg/mL sodium bicarbonate (Sigma S5761), 10 mM HEPES (Gibco 15630106), 5% FBS (Gibco, 16000044), 1% NEAA (Multicell 321-011-EL), 25 U/mL streptomycin-penicillin (Gibco 15140-122) which was supplemented with 0.1 mM Luciferin. Then plates were sealed with silicone grease to prevent evaporation and immediately placed in LumiCycle. This instrument continuously recorded the luminescence for each plate for 5-7 days with 10 minutes intervals at 37° C. LumiCycle Analysis software (Actimetrics) was used to analyse the changes in circadian rhythm. The data of the first day was excluded from analysis due to transient deviations caused after medium change.

For the experiments that were performed using Synergy-H1, U2OS Bmal1-dLuc cells were plated as triplicates into an opaque 96-well plate (5×104 cell/well). After synchronizing and treating the cells with CLK95, opaque 96-well plate was sealed with an optically clear film and placed under Synergy-H1 instrument. Luminescence signals were recorded for 5-7 days with 24 minutes intervals at 32° C.

### Example 6 Lentivirus Production and Transduction

Wild type (WT), Clock knockout (KO), and Bmal1 KO mouse embryonic fibroblast (MEF) cells were transduced with Bmal1-dLuc lentiviral particles to enable us to monitor the effect of CLK95 on circadian oscillation. To this end, virus particles were produced as follow: HEK 293T cells were transfected with 10 μg pLV6-Bmal1-dluc, 9 μg pCMV-ΔR8.2dvpr packaging and lug pCMV-VSVG envelope vectors at ˜70-80% confluency. The plasmid cocktail was mixed with 20 μL PEI in 400 μL DMEM and incubated at room temperature for 10 minutes and cells were transfected. After 16 hours of incubation at 37° C. under 5% CO2, 3 mL fresh D10 medium were added to plates. 48 hours after transfection, cell medium was replaced with 8 mL fresh D10 medium. Lentiviral particles were harvested at 72 and 96 hours post transfection. Collected particles were filtered through 0.45 μM syringe filter and aliquoted (750 μL) for storage at −80° C. for further use.

WT, Clock KO, and Bmal1 KO MEF cells were transduced with Bmal1-dluc lentiviral particles as follow: Cells were seeded in 35 mm cell culture plates at the density of 1×105 cell/plate. Next day, cells were transduced with lentiviral particles by replacing the cell medium with virus-containing master mix which was prepared by mixing the virus-containing aliquots (750 μL) with 250 μL DMEM and 8 μg protamine sulphate. 16-hours after incubation, cell media was replaced with 1.5 mL fresh D10. 72-hours post-transduction cells were synchronized with Dexamethasone (0.1 μM) and prepared for luminescence recording of the circadian oscillation as indicated at section 3.4

### Example 7 Pull-Down Assay

To perform pull-down assay HEK 293T cells were transiently transfected with either 10 μg pCMV-Sport6-CLOCK or 10 μg pCMV-Sport6-Bmal1 plasmids. First, cells were seeded in a 10 cm cell culture dish at a density which will give ˜70-80% confluency next day. Then cells were transfected with 10 μg of the appropriate plasmid using 30 μL PEI in 1 mL DMEM. Cells were harvested 24 hours post-transfection and divided into four 1.5 mL centrifuge tubes. The conditions that were employed to probe the binding of the molecule to proteins contained a) only CLOCK overexpressed, b) only BMAL1 overexpressed and c) CLOCK-BMAL1 overexpressed mixtures. Pellet for each condition was lysed with 500 μL Lysis Buffer (50 mM Tris-HCl PH 7.4, 2 mM EDTA, 1 mM MgCl2, 0.2% NP-40, 1 mM Na3VO4, 1 mM NaF, 1×PIC) and incubated on ice for 20 minutes. Then samples were centrifuged at 7000×g for 10 minutes at 4° C. The supernatants of the CLOCK and BMAL1 lysates were mixed at 1:1 ratio. Following this step, collected lysate mixture was mixed with 2× binding buffer (100 mM Tris-HCl pH 7.4, 300 mM NaCl, 0.2% NP-40, 2 mM Na3VO4, 2 mM NaF, 2×PIC) at 1:1 ratio. While 5% of the mixture was kept as input, the remaining part was divided equally into three separate microcentrifuge tubes and incubated by continuous mixing with either DMSO, or 20 μM biotinylated-CLK95 (bCLK9) with or without 200 μM non-biotinylated CLK95 (competitor) at 4° C. for 90 minutes. Meanwhile, the NeutrAvidin Agarose resin (Thermo Scientific 29201) was equilibrated (30 μL settled resin/sample). Equilibration was performed according to manufacturer's protocol. Lysates were supplemented with equilibrated NeutrAvidin resin and incubated for 24 hours at 4° C. with constant shaking. The next day, samples were centrifuged at 2500×g for 2 minutes and then supernatants were discarded. The resin of each sample was washed 3 times with 1× binding buffer for 5 minutes with constant mixing at 4° C. Subsequently, resin of each sample was boiled with 1× Laemmli buffer for 7 minutes at 95° C. for SDS-PAGE analysis. After samples subjected to the SDS-PAGE, proteins were transferred to PVDF membrane (Millipore) for Western blot study. Upon completion of transferring proteins, the membrane was incubated with 5% milk powder in 0.15% TBS-T for an hour. Then the membrane was incubated with anti-CLOCK (Bethyl, A302-618A) and anti-BMAL1 (Santa Cruz Biotechnology, SC-365645) antibodies diluted in 5% milk powder in 0.15% TBS-T. Finally, membrane was washed three times with TBS-T and incubated with secondary antibodies conjugated wit with HRP. Membranes were subjected to homemade ECL, and images were taken with chemiluminescence (Bio-Rad).

### Example 8 Site Directed Mutagenesis

To investigate the binding sites of the CLK95 on CLOCK and BMAL1, promising residues which were computationally determined to interact with small molecule were mutated via site-directed mutagenesis. Computational study revealed that R126 of the BMAL1 and F80-K220 residues of the CLOCK were important for the binding of the CLK95. These amino acid residues were introduced to Bmal1 and CLOCK genes by PCR based mutagenesis. FLAG-tagged pcDNA4a-Bmal1 and pCMV-Sport6-Bmal1 were used as template DNAs. Each PCR reaction consisted of 50 ng of template DNA, 0.2 μM primer pair, 0.2 mM dNTPs and 3 units of Pfu DNA polymerase. Then the PCR reaction was performed under these conditions: 5 minutes at 98° C. followed by 12 cycles of 1 minute at 98° C., 1 minute at 55° C. and 15 minutes at 72° C. The PCR reaction completed with a final extension step at 72° C. for 15 minutes. PCR products were analysed with 0.8% of agarose. Bands at the right size were further treated with 1 μL FastDigest Dpn I (Thermo Scientific, FD1704) for 1 hour at 37° C. to digest methylated-parental DNA. Subsequently, DH5a (a strain of E. coli) cells were transformed with the 7 μL PCR product. Standard heat-shock transformation protocol was used. Transformed cells were plated on Ampicillin (100 μg/mL) containing Luria-Bertani (LB) agar plates and incubated overnight at 37° C. Later, colonies formed on the LB ager plate were selected to culture in 5 mL LB broth containing Ampicillin (100 μg/mL). After overnight incubation at 37° C., cells were harvested and plasmid isolation was performed with Macherey Nagel's NucleoSpin Plasmid (NoLid), Mini kit. Isolated plasmids were then sent for Sanger sequencing (Macrogen) to confirm desired single-site mutation.

### Example 9 Co-Immunoprecipitation (Co-IP)

To assess the effect CLK95 on BMAL1-CLOCK interaction Co-IP was employed. HEK 293T cell were transfected with FLAG-tagged pcDNA4a-Bmal1 or pCMV-Sport6-CLOCK plasmids. The FLAG-tagged pcDNA4a-Bmal1-R126A and pCMV-Sport6-CLOCK-F80A-K220A mutants were used in this assay. The R126 residue in the BMAL1 and F80-K220 residues in the CLOCK were identified to be important for the interaction of CLK95 by computational analysis. Therefore, these mutations were used as negative control during the Co-IP studies. After the transfection cells were harvested and were lysed with 500 μL Lysis buffer (50 mM Tris-HCl pH 7.4, 150 mM NaCl, 0.1% Triton X-100, 1×PIC). After centrifugation at 15000×g for 20 minutes, supernatants were transferred into new tubes. The protein concentration of each lysate was determined with the Pierce™ 660 nm Protein assay (Thermo Scientific, 22660). For each fraction 150 μg protein was used with a 2:1 ratio of CLOCK and BMAL1 proteins, respectively. CLOCK and BMAL1 lysates were and divided into two fractions which were treated with DMSO and CLK95. BMAL1-FLAG and BMAL1-R126A-FLAG were precipitated with Anti-Flag M2 Affinity Gel (Sigma-Aldrich, A2220). Then, the bound proteins were eluted with 30 μL 4× Laemmli buffer at 95° C. for 7 minutes after washing the resin with 1×TBS buffer (3 times). After quick centrifuge, 15 μL of each sample was subjected to 8% SDS-PAGE following by Western blot as described above using anti-CLOCK and anti-FLAG (Sigma, A9469-1 MG) antibodies.

### Example 10 Transactivation Assay

To confirm Bmal1-R126A mutation has no effect on its binding to CLOCK, transactivation assay was employed. HEK 293T cells were transiently transfected via reverse transfection with 125 ng pCMV-Sport6-CLOCK, 50 ng pGL3-Per1::Luc and 50 ng pcDNA4a-Bmal1 (wild type) or 50 ng pcDNA4a-Bmal1 (R126A) and 3 ng of pGL4-Renilla (for normalization). Cells were seeded as triplicates into an opaque 96-well plate (4×104 cells/well) and maximum 300 ng plasmid was used for each condition (completed with 72 ng pCMV-Sport6 plasmid). 19 hours after incubation at 37° C. under 5% CO2, cells were lysed with Firefly Luciferase buffer and measured by Fluoroskan Ascent. (Final concentration: 5 mM DTT, 0.2 mM Coenzyme, 0.15 mM ATP, 1.4 mg/mL Luciferin, 0.03M Tris-HCl, 0.01M Tris-base, 25 mM NaCl, 1 mM MgCl2, 0.08% Triton X-100). After recording the firefly luciferase activity, Renilla luciferase activity was measured by incubating the mixture with 1× Renilla luciferase buffer which was diluted from main stock of 3× Renilla luciferase buffer (0.06 mM PTC124 (in DMSO), 0.01 mM h-CTZ (in ethanol), 45 mM Na2EDTA, 30 mM Na4P207, 1.425M NaCl) for 10 minutes at room temperature. Relative luciferase activity (RLU) which was normalized to Renilla was calculated according to Equation 3.2.

$$\begin{matrix}Equation 3.2 . RLU calculation formula & \\ RLU = \frac{Average ( \frac{Firefly}{Renilla} ) sample}{Average ( \frac{Firefly}{Renilla} ) DMSO} . & \end{matrix}$$

### Example 11 the Effect of CLK95 on Core Clock Protein

The effect of CLK95 on core-clock proteins was assessed both on synchronized and unsynchronized U2OS Bmal1-dLuc cells. Cells were seeded into 6 well-plates with a density of 45×104 cell/well and incubated at 37° C. under 5% CO2 for 16 hours. For unsynchronized cells, they were treated with appropriate amounts CLK95 (final 0.25% DMSO) and further incubated for 24 hours. For synchronized cells, cell media was exchanged with 0.1 μM Dexamethasone containing DMEM. After synchronization, cells were subjected to the CLK95 (final 0.25% DMSO). After 24 hours of incubation, cells were harvested with 6 hours intervals for 7 time points (24H-60H). Then harvested cells were lysed with 90 μL RIPA buffer (50 mM Tris-HCl pH 7.4, 150 mM NaCl, 1% Triton x-100, 0.1% SDS, 1×PIC) and left for an incubation on ice for 30 minutes. Following, samples were centrifugated at 15000×g for 20 minutes at 4° C. After boiling samples with Laemmli buffer for 7 minutes at 95° C., 10 μg of protein was loaded for each sample to 10% SDS-polyacrylamide gel. For Western blot analysis following antibodies used to determine the level of core clock proteins: anti-CLOCK (Bethyl, A302-618A), anti-BMAL1 (Santa Cruz Biotech., SC-365645), anti-PER2 (Proteintech, 67513-1-Ig), anti-CRY1 (Bethyl, A302-614A), and anti-β-Actin (Cell Signaling, 8H10D10) antibodies.

### Example 12 Total RNA Isolation and RT-qPCR

The effect of small molecule on core-clock genes were explored through RT-qPCR. TRIzol reagent (Invitrogen, Cat. No. 15-596-018) was used for RNA isolation from unsynchronized and synchronized U2OS Bmal1-dLuc cells. After resuspending cells in 1 mL TRIzol reagent, 200 μL chloroform was added to each sample. Then the mixture was incubated at room temperature for 5 minutes. Samples were centrifugated at 14000×g for 20 minutes at 4° C. after completing the incubation phase. Newly formed aqueous phase was transferred to a new microcentrifuge tube and mixed with 1:1 ratio with isopropanol and 12 μg glycogen for each sample. After that, mixtures were incubated at −80° C. for 1.5 hours. Next, samples were centrifugated at 14000×g for 20 minutes (4° C.) and the pellets that were formed at the bottom of the tubes were washed with 1 mL of 75% ethanol for 3 times. At the end of each wash step, centrifugation at 14000×g for 5 minutes (4° C.) was done. Supernatants were discarded and the pellets were left for air-dry at room temperature following last wash. Then, RNA pellets were dissolved in 30 μL nuclease-free water. Quantity and the quality of RNAs were controlled with Nanodrop 2000 (Thermo Scientific) and agarose gel electrophoresis, respectively. Agarose gel electrophoresis was performed at 100 Volt (V) for 15 minutes using 0.8% agarose gel which was prepared with DEPC (diethylpyrocarbonate) treated water.

Following RNA isolation, total RNAs were converted into cDNA using Thermo Scientific's RevertAid First Strand cDNA Synthesis Kit as described in product's manual. Each reaction consists of 0.5 μg RNA template, 5 μM Oligo (dT) 18 primer, 1× Reaction Buffer, 1 U/μL RiboLock RNase Inhibitor, 1 mM dNTP mix, and 10 U/μL RevertAid M-MuLV RT. Then, For Real-Time PCR (qPCR), cDNAs were further diluted with nuclease-free water and the qPCR experiments were conducted with 1:40 diluted cDNAs.

For qPCR assay, reaction mixture containing 10 μL 2× SensiFAST SYBR® No-ROX Mix (Bioline), 1 μL forward and reverse primer mix (final concentration 0.5 μM from each), 6 μL nuclease-free water, and 3 μL 1:40 diluted cDNA was prepared for each sample. The complete list of the primers is given in Appendix B. RPLP0 (ribosomal protein, large, P0) was used as the reference gene. After qPCR reaction mixtures were prepared, following cycling protocol was performed using CFX Connect Real-Time PCR detection system (Bio-Rad). 7 minutes of initial denaturation at 95° C. followed by 40 amplification repeats which subsequently contained, 8 seconds at 95° C., 10 seconds at 60° C. and 20 seconds at 72° C. At the end of each amplification step, fluorescence signals were recoded. After completing amplification steps, reaction mixtures were incubated 8 seconds at 95° C., 5 seconds at 70° C., and 5 seconds at 90° C., respectively. Last fluorescence recording was done in this step after incubating at 70° C. for 5 seconds. The results were analyzed with the delta-delta Ct (2-ΔΔCt) method.

### Example 13 Cycloheximide (CHX) Chase Experiment

To investigate if the small molecule has any effect on stability of CLOCK and BMAL1, Cycloheximide-chase experiment was conducted. At first day, HEK 293T cells were seeded into 6-well plates with a density of 37.5×104 cell/well. Next, these cells were transiently transfected with 65 ng FLAG-tagged pCMV-Sport6-CLOCK, 875 ng FLAG-tagged pcDNA4a-Bmal1, and 60 ng pCMV-Sport6 by 3 μL PEI. 24 hours after transfection, cells were treated with either DMSO (0.25%) or 5 μM small molecule. 48 hours after transfection, cells were treated with 100 μg/mL CHX to stop protein synthesis. Following CHX treatment cells were harvested with 3 hours intervals for 12 hours. Cells harvesting and protein isolation were executed as previously described in Protein isolation and Western blotting section. Selective immunodetection was performed via anti-FLAG (Sigma-Aldrich, A9469-1 MG) and anti-β-Actin (Cell Signaling, 8H10D10) antibodies.

### Example 14 Subcellular Fractionation

Subcellular fractionation was performed to explore if CLK95 effects the subcellular localization of core-clock proteins. U2OS Bmal1-dLuc cells were plated with a density of 40×104 cell/well in 6 well plate, a day prior to small molecule treatment. Following, cells were treated with either DMSO (0.25%) or 5 μM CLK95. 24 hours after small molecule treatment, cells were harvested to get fractionated. Cells were harvested with 1 mL ice cold-1×PBS, and pelleted at 5000×g for 7 minutes at 4° C. After discarding supernatants, cells were resuspended with 200 μL Cytosolic Lysis Buffer (CLB) (10 mM HEPES pH 7.9, 10 mM KCl, 0.1 mM EDTA pH 8.0, 0.05% NP-40, 1×PIC) and incubated on ice for 10 minutes. Then, samples were centrifuged at 660×g for 5 minutes (4° C.). While supernatants were transferred to new centrifuge tubes as cytosolic fractions, pellets were washed with 200 μL CLB buffer for 3 times. At the end of each wash, samples were centrifugated at 660×g for 5 minutes at 4° C. Next, cell pellets were resuspended in 100 μL Nuclear Lysis Buffer (NLB) (20 mM HEPES pH 7.9, 0.4M NaCl, 1 mM EDTA, 10% Glycerol, 1×PIC) and subjected to sonication (60% power, 10 seconds, 3 cycles) (BANDELIN SONOPULS HD 2070 homogeniser) for 2 times. This step was followed by centrifugation at 15000×g for 5 minutes with the cytosolic fractions. Each cytosolic and nuclear fractions were transferred into new tubes and subjected to the Western blotting analysis. For Western blot analysis following antibodies used: anti-CLOCK (Bethyl, A302-618A), anti-BMAL1 (Santa Cruz Biotech., SC-365645), anti-PER2 (Proteintech, 67513-1-Ig), anti-CRY1 (Bethyl, A302-614A) anti-Histone H3 (Abcam, ab1791), and anti-αTUBULIN (Sigma-Aldrich, T9026) antibodies. While Histone H3 was used as an internal control for normalization of nuclear fractions, αTubulin was used for normalization of cytosolic fractions.

### Example 15 Maintenance of Animals

8-12 weeks old male C57BL/6J mice were used for in vivo studies. Mice weighing 24-28 grams were subjected to a single dose of CLK95 (50 mg/kg) or vehicle (DMSO:Cremophor EL: 0.9% NaCl; 2.5:15:82.5, v/v/v) intraperitoneally. Animals which were obtained from Koç University Animal Research Facility were fed ad libitum. Four hours after treatment, animals were sacrificed, and the internal organs were harvested.

### Example 16 Protein Isolation and Western Blot Analysis from Mice Liver

Mice (C57BL/6J) treated with either vehicle or CLK95 (50 mg/kg) were sacrificed 4 hours post-treatment to study the effect of molecule in vivo. Liver tissue was collected and snap-frozen in liquid nitrogen to further investigate the protein and RNA expression levels. Snap-frozen samples either immediately used or stored in −80° C. for long term usage. To isolate proteins, 10 mg liver tissue was dissected and washed with 500 μL 1× ice cold PBS buffer. Dissected tissue then, homogenized for 2 minutes on ice using 500 μL RIPA buffer (recipe is given in section 3.10.). After incubating the suspension for 20 minutes on ice, samples were centrifugated at 10000×g for 30 minutes at 4° C. Supernatants were harvested and transferred to new microcentrifuge tubes to perform Pierce 660 nm Protein Assay to measure protein concentrations. Samples that were assayed, supplemented with Laemmli buffer to perform SDS-PAGE and Western-Blot techniques. 10 μg from each sample was used. For immunodetection, anti-CLOCK, anti-BMAL, anti-PER2, anti-CRY1, and anti-ACTIN antibodies were used.

### Example 17 RNA Isolation and RT-qPCR Analysis from Mice Liver

RNA isolation from mice liver was operated according to RNeasy® Mini Kit from Qiagen (Cat no. 74106). After RNAs were eluted in 50 μL nuclease-free water, quantity, and the quality of RNAs were assessed with Nanodrop 2000 (Thermo Scientific) and agarose gel electrophoresis, respectively. Following RNA isolation, cDNA synthesis and qPCR assay performed as previously described in section 3.11.

### Example 18 Subcellular Fractionation from Mice Liver

Subcellular fractionation from mice liver was performed by using 10 mg tissue sample. Buffers that were used in this process are same as in given in section 3.13. First, the tissue was washed in 500 μL ice-cold 1×PBS buffer and then resuspended in 300 μL cytosolic lysis buffer by pipetting and incubated on ice for 10 min. Samples were centrifuged at 660×g for 5 minutes (4° C.) to get rid of cell debris. While supernatants were collected and transferred to new microcentrifuge tubes for processing later to give cytosolic fractions, pellets containing intact nucleus were washed 3 times with 300 μL cytosolic lysis buffer. At the end of each wash, suspensions were centrifugated for 5 minutes at 660×g (4° C.). Pellets were resuspended in 200 μL nuclear lysis buffer and sonicated two times with 60% power for 10 seconds with 3 cycles. Supernatants were collected and labelled as cytosolic and nuclear fractions depending on the buffer they were dissolved. Samples were analysed with Western blot as described earlier using anti-CLOCK (Bethyl, A302-618A), anti-BMAL1 (Santa Cruz Biotech., SC-365645), anti-CRY1 (Bethyl, A302-614A), anti-Histone H3 (Abcam, ab1791), and anti-αTUBULIN (Sigma-Aldrich, T9026) antibodies were used.

### Example 19 Statistical Analysis

All data were represented as mean±SEM (Standard Error of Mean) and statistical significance was evaluated with Student's t-test by using GraphPad Prism (GraphPad Software, California, USA). For each experimental setup, number of biological replicates was at least n=3. [*p-value<0.05, **p-value<0.01, ***p-value<0.001.]

What is claimed is:

1. A circadian locomotor output cycles kaput: brain and muscle arnt-like 1 (CLOCK:BMAL1) complex-binding compound, comprising the following formula:

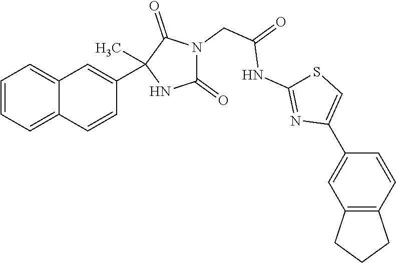 [SMILES: CC1(c2ccc3ccccc3c2)NC(=O)N(CC(=O)Nc2nc(-c3ccc4c(c3)CCC4)cs2)C1=O]

2. A method for treating and/or preventing a disease or disorder associated with circadian rhythm, comprising using the CLOCK:BMAL1 complex-binding compound according to claim 1.

3. A method for treating and/or preventing a disease or disorder due to an increase of CLOCK:BMAL1 interaction and stabilization of a CLOCK:BMAL1 complex, comprising using the CLOCK:BMAL1 complex-binding compound according to claim 1.

4. The method according to claim 2, wherein the disease or disorder is a sleep disorder, mood disorder or aging and diseases related with an altered circadian amplitude.

5. The method according to claim 4, wherein the disease or disorder is the sleep disorder.

6. A preparation method of a medicament for a treatment and/or prevention of a disease or disorder associated with circadian rhythm, comprising using a compound having the following formula or a pharmaceutically acceptable salt thereof,

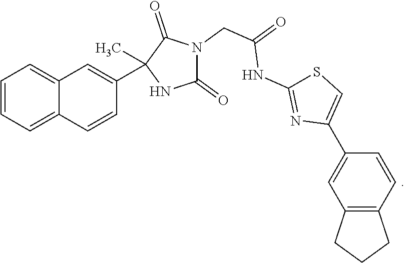 [SMILES: CC1(c2ccc3ccccc3c2)NC(=O)N(CC(=O)Nc2nc(-c3ccc4c(c3)CCC4)cs2)C1=O]

7. The preparation method according to claim 6, wherein the medicament is useful in a treatment and/or prevention of a disease or disorder due to an increase of CLOCK:BMAL1 interaction and stabilization of a CLOCK:BMAL1 complex.

8. The preparation method according to claim 7, wherein the disease or disorder associated with the circadian rhythm and the disease or disorder due to the increase of the CLOCK:BMAL1 interaction and the stabilization of the CLOCK:BMAL1 complex are a sleep disorder, mood disorder or aging and diseases related with an altered circadian amplitude.

9. The preparation method according to claim 8, wherein the disease or disorder associated with the circadian rhythm and the disease or disorder due to the increase of the CLOCK:BMAL1 interaction and the stabilization of the CLOCK:BMAL1 complex are the sleep disorder.

10. The preparation method according to claim 9, wherein the sleep disorder is selected from jet lag syndrome, shift work sleep disorder, advanced sleep phase syndrome, non-24-hour sleep-wake syndrome, irregular sleep-wake rhythm, and delayed sleep phase syndrome.

11. A pharmaceutical composition comprising a pharmaceutical carrier and a therapeutically effective amount of the CLOCK:BMAL1 complex-binding compound according to claim 1, or a pharmaceutically acceptable salt thereof.

12. A method for identifying a compound increasing an interaction between CLOCK and BMAL1, wherein the method comprises contacting the compound with a CLOCK:BMAL1 complex under conditions allowing for an interaction of the compound with the CLOCK:BMAL1 complex, and determining whether the compound increases the interaction between the CLOCK and the BMAL1 by using a system, wherein the system uses a signal and/or a marker generated by the interaction between the CLOCK and the BMAL1 to detect a presence or absence or change of the signal and/or the marker.

13. The method according to claim 3, wherein the disease or disorder is a sleep disorder, mood disorder or aging and diseases related with an altered circadian amplitude.

14. The method according to claim 13, wherein the disease or disorder is the sleep disorder.
